(19) United States
(12) Patent Application Publication
(10) Pub. No.: US 2006/0173360 A1
(43) Pub. Date: Aug. 3, 2006

(54) Method for detection and display of extravasation and infiltration of fluids and substances in subdermal or intradermal tissue

(76) Inventors: John F. Kalafut, Oakmont, PA (US); David M. Griffiths, Pittsburgh, PA (US); Arthur E. Uber III, Pittsburgh, PA (US)

Correspondence Address: James R. Stevenson, Esquire;MEDRAD, INC., One Medrad Drive, Indianapolis, PA, 15051, US

(21) Appl. No.: 11/031,897

(22) Filed: Jan. 7, 2005

(51) Int. Cl. A61B 6/00

(52) U.S. Cl. 600/478

(57) ABSTRACT

A method for the real-time visualization and detection of extravasated and or infiltrated fluid and substances, including blood, that occur near the cannulation site of an injection is described wherein illumination or transillumination with near infrared light is used to image the contrast in real-time between absorbing and nonabsorbing subdermal and intradermal structures of blood vessels and remaining surrounding tissue, foreign substances and other structures in order to establish a baseline image of the body area of interest, and any new image is monitored and compared with the baseline image to detect the extravasation and/or infiltration of fluids and substances, including blood, around a vein or artery into the subdermal or intradermal tissue.

### FIELD OF THE INVENTION

The invention relates generally to medical devices and procedures, and more particularly to procedures for the real-time detection and visualization of infiltrated or extravasated substances, including blood, that occur in subdermal or intradermal tissues near the cannulation site of an injection procedure, such as in the intravascular delivery or extraction of various substances or media.

### BRIEF DESCRIPTION OF RELATED ART

The following information is provided to assist the reader to understand the invention disclosed below and at least some of the applications in which it will typically be used. In addition, any references set forth herein are intended merely to assist in such understanding. Inclusion of a reference herein, however, is not intended to constitute an admission that the reference is available as prior art with respect to the invention.

During intravascular administration of substances, a portion of the substances may escape from the interior of the vein or artery into surrounding tissues. Leaking of intravascular fluids or medicine is referred to as infiltration if the substance is limited to causing mild effects such as swelling and may include bleeding. Extravasation describes leaking of intravascular fluids or medicines that may, in a worst case scenario, cause tissue damage. Bleeding may also be described as extravasation as it indicates some rupture or failure of the vessel wall(s). Incidents that cause extravasation and infiltration include improper venipuncture, such as a transfixation of a vein, rupture of the vasculature, perhaps due to weakened vascular walls in patients of advanced age, disease states, abrasion by the cannula, or the administration of a toxic agent. Problems with extravasation and infiltration may include bruising, discoloration of the skin or discomfort to the patient, or more serious problems associated with thrombosis, bleeding leading to hypovolemia, and tissue toxicity, such as in the administration of toxic substances associated with chemotherapy wherein the concentration of a toxic substance is carefully monitored to ensure dilution of the substance to appropriate levels during administration. Extravasation into the spaces surrounding a blood vessel poses very serious problems of local tissue toxicity and/or possible necrosis depending upon the agent, and a lack of accurate delivery of metered dosage into the patient's vascular system.

Treatment of suspected extravasation may be costly and time consuming. First, an effort must be made to determine whether an extravasation or infiltration has occurred, the material injected, the injection location, and the amount of injected volume into the tissue. If the amount injected is excessive or there is an injection of toxic materials, the extravasate fluid must be removed through a surgical drainage procedure, otherwise the patient is monitored over time until the swelling and risk of adverse effects decrease sufficiently. Often this involves additional medical attention and extra time spent in the medical clinic or hospital under medical care.

Non-invasive detection of extravasation and/or infiltration in the prior art is accomplished chiefly by medical personnel visualizing a swelling in the region with an unaided eye or manually palpating the region or by assessment of patient's complaint of discomfort, pressure, swelling, or pain. Often, during diagnostic imaging procedures, where large amounts of fluid are injected, a test injection is performed first to verify venous access and proper catheter placement to confirm that the fluid from the outlet of the catheter is flowing correctly into the injected vessel. Alternatively, the first several seconds of an injection are monitored manually. In such cases, large amounts of extravasated fluids and/or blood components may accumulate in the tissue surrounding the vasculature before detection of the condition is made.

Other automatic methods exist for detection of extravasation and/or infiltration using various electronic signal and detection systems. One such method found in U.S. Pat. No. 4,877,034 to Atkins et al., a photo-plethysmographic technique, discloses a method for detecting a plurality of wavelengths of electromagnetic radiation emitted and then detected as reflected or reemitted radiation at the injection site. Such measurements are taken prior to or in the absence of injection in order to establish a baseline. During injection, infiltrated or extravasated fluids cause a shift in the detected radiation that can be measured against the baseline for evidence of infiltration or extravasation.

Another method, disclosed in U.S. Pat. No. 4,647,281 to Carr, involves subcutaneous temperature sensing via an antenna and a microwave radiometer and measurement of the temperature of fluid introduced at an injection site. In this method, an alarm is activated when the temperature differential between the injected fluid and the surrounding tissue reaches a prefigured threshold. Yet, other detection techniques rely upon plethysmographic measurements of volume changes in the tissue surrounding an injection and extravasation and/or infiltration site. Changing volumes of subdermal fluid resulting from infiltration may be measured as changes in tissue impedance, deflections of strain cuffs, or changes in pressure sensors at the site.

These prior methods as a whole exhibit significant limitations in that they cause direct obstructions at the site of injection and extravasation or infiltration thereby preventing visualization and palpation of the tissue, a critical step by the healthcare provider under all circumstances. As an example, U.S. Pat. No. 4,877,034 requires placement of an electromagnetic radiation emitter and detector patch directly over the area of injection and accordingly prevents observation and palpation of the physical signs of extravasation or infiltration.

One method that avoids obstruction of observation and palpation is disclosed in U.S. Pat. No. 6,459,931 to Hirschman. This method provides for placement of a first order energy source and receiver in close proximity to the injection site; the receiver measures changes in bulk electrical properties of the tissue and fluid at the infiltration site. This method, along with the other prior art for detection of extravasation, only discloses a technique for generation of electronic signals for processing and interpretation of extravasation or infiltration events; they do not teach a method of immediate visual display of the actual physical features in the form of contrast images of the extravasating or infiltrating fluids. An unobstructed visual display of extravasation or infiltration offers the primary advantages of instant confirmation and assessment by the healthcare provider including indications of volume, rate, and distribution of extravasating fluids in complement to palpation of the site.

Prior art methods to assist medical personnel in the visualization of veins, arteries and other subcutaneous structures of the body include application of tourniquets, use of a flashlight, direct application of liquid crystalline materials, ultrasound and use of dual fiber optic sources. These methods may indicate vein or artery location, but do not allow for the detection of extravasation or infiltration in direct visual respect to the vasculature. A procedure is therefore needed to detect reliably and non-invasively and to display extravasation and infiltration in real-time, especially at the time a patient is undergoing a procedure, in order to identify and to diagnose the extravasation or infiltration of blood components or other fluids and substances, such as injectable agents, at the time of administration.

Transillumination and reflection imaging in the near infrared (NIR) are non-invasive techniques for detecting the vasculature wherein the passage of light through the body or reflection from its surface and near subsurface regions are used to observe subsurface structures. Using such techniques, a body surface area of interest is illuminated and characteristics, such as light intensity and wavelength reflected or scattered, from that area form an image. U.S. Pat. No. 6,230,046 to Crane et al. ('046 patent) teaches a device that illuminates veins and arteries and displays blood vessel structure in-vivo in a non-invasive and painless manner to facilitate insertion or extraction of fluids for medical treatment. However, the device and method taught in the '046 patent does not address the sensing, and display the presence, of extravasated or infiltrated fluids and substances into subdermal or intradermal tissues. The entire teachings of the '046 patent are incorporated herein by reference.

To overcome the limitations of such prior art techniques, it would be desirable to provide an easily transportable, non-invasive, real-time method for detecting and displaying extravasation or infiltration of subdermal and/or intradermal tissue without direct obstruction at the site of injection and suspected infiltration or extravasation. In accordance with such a method, differences in image content would be significant and would permit detection and visualization of extravasation or infiltration of contrast media, blood, medicines, or other fluids and substances; the degree of contrast in the NIR would be used to quantify the amount of nonabsorbing or absorbing substance being infused and subsequently leaking from the intended vessel.

It would also be desirable to employ illumination or transillumination with NIR to image the contrast in real-time between absorbing and nonabsorbing subdermal and intradermal structures of blood vessels and remaining surrounding tissue, foreign substances and other structures. After baseline images have been established, any new image would be monitored and compared with the original to detect the extravasation or infiltration of fluids or blood around a vein or artery and into the subdermal and/or intradermal tissues. Such a desired invention would allow the detection of location, size, depth, direction of movement or flow, rate of movement or flow, shape, constitution, volume of features or other defining aspects.

Such an invention would find substantial use in numerous medical procedures. Examples of such procedures include demonstrating the proper technique for inserting an IV catheter, avoiding severe bruising during IV access for medical treatment, and correctly administering various medications and imaging contrast agents. Other examples include monitoring arterial bleeding after removal of a femoral artery catheter and avoiding thrombosis, and monitoring rapid extravasation of high-pressure, injected contrast dyes following vessel rupture.

It is therefore an objective of the invention to provide a system and a method for real-time detection and visualization of extravasation and infiltration of fluids and substances in subdermal or intradermal tissues.

It is another objective of the invention to provide a method for detection of extravasation and infiltration during a medical procedure.

These and other objectives of the invention will become apparent as the detailed description of representative embodiments proceeds.

### SUMMARY OF THE INVENTION

The foregoing objectives and advantages of the invention are attained by the embodiment(s) and related aspects of the invention summarized below.

In accordance with the foregoing principles and objectives of the invention, a method for the real-time visualization and detection of extravasated or infiltrated fluids and substances, including blood, that occur near the cannulation site of an injection is disclosed wherein illumination or transillumination with NIR or other wavelengths of the infrared band is used to image the contrast in real-time between absorbing and nonabsorbing subdermal and intradermal structures of blood vessels and remaining surrounding tissue, foreign substances and other structures in order to establish a baseline image of the body area of interest, and any new image is monitored and compared with the baseline image to detect the extravasation or infiltration of fluid and substances, including blood, around a vein or artery and into the subdermal or intradermal tissue.

### BRIEF DESCRIPTION OF THE DRAWINGS

The invention will be better understood by reference to the detailed description below and to the accompanying drawings, in which:

FIG. 1 shows schematically a light source, detector, a forearm of a patient, and an IV instrument illustrating an embodiment of the invention;

FIG. 2a shows schematically a baseline image of the forearm of the patient as in FIG. 1;

FIG. 2b shows schematically the baseline image of FIG. 2a after a substance transparent to NIR is inserted into a vein of the patient as in FIG. 1;

FIG. 2c shows schematically the baseline image of FIG. 2a after a substance absorbent of the NIR is inserted into a vein of the patient; and

FIG. 3 shows schematically the forearm of a patient of FIG. 1 after the IV instrument is removed illustrating the presence of extravasation or infiltration around a vein.

FIG. 4 shows the mid-infrared spectrum of diatrizoic acid, an ingredient in some imaging contrast agents.

### DETAILED DESCRIPTION OF THE INVENTION

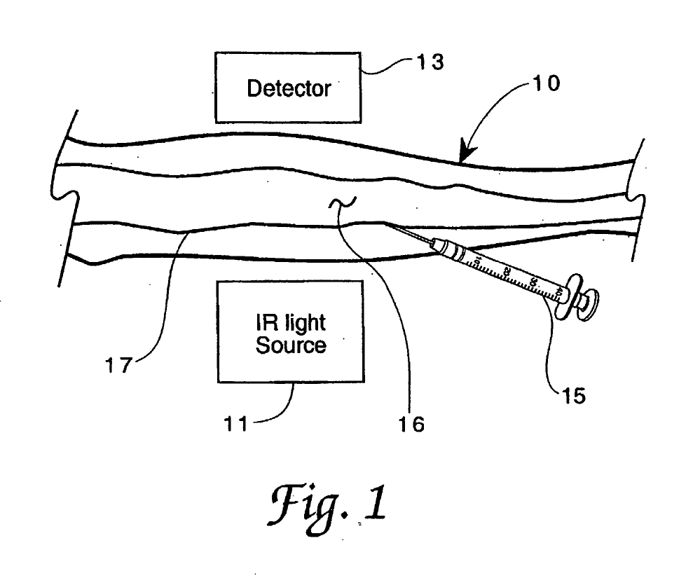

Referring now to the drawings, FIG. 1 illustrates the method of the invention in one of its embodiments for imaging extravasation or infiltration, wherein a surface area of the body, such as forearm 10, is placed near an NIR light source 11 and between source 11 and detector 13 (in the transillumination mode of the invention), in order to perform a particular procedure, such as in the administration of a specific substance into the vasculature (artery or vein 17) using IV instrument 15. Source 11 preferably emits light in the wavelength range of about 0.3 to 1.0 microns (μm), and detector 13 is sensitive to light in that range. Alternatively, source 11 could be placed to directly illuminate forearm 10 such that a reflected NIR image of the infusion area 16 at instrument 15 is viewed. Source 11 can also be placed near the area of interest of forearm 10 such that scattered light is used to illuminate the area of interest. Detector 13 is focused upon the area of interest and does not interfere with the procedure or obstruct medical personnel performing the procedure. NIR energy from source 11 and detected by detector 13 may be that reflected, refracted, absorbed, transmitted or scattered by subdermal and intradermal structures in the body area of interest, such as forearm 10.

Detector 13 may include any of the instruments well known in the art and used for acquiring and displaying NIR images of the body area of interest, such as those described in the '046 patent, including image intensifier tubes (night vision goggles), photomultiplier tubes, photodiodes, silicon based arrays such as charged couple devices (CCD), complementary metal-oxide semiconductor (CMOS), or other solid state devices with appropriate filtering to enhance the signal-to-noise ratio of the image. Detector 13 converts the NIR image to a visible one so that medical personnel may view the infusion procedure in order to properly insert instrument 15 and to detect any extravasation or infiltration of the infusion fluid or substance, including blood, that occurs in the area of interest. The detected energy is selectively filtered to eliminate light from interfering sources and to create one or more images of subcutaneous structures in the area. In a preferred embodiment, the detected energy is selectively filtered using a filter having a narrow passband centered substantially on at least one wavelength in the range of about 0.30 to 1.0 μm and more particularly at wavelengths of about 0.32, 0.345, 0.41, 0.43, 0.455, 0.54, 0.56, 0.58, 0.7, 0.76 μm. The image generated from the filtered light is used to determine inherent, baseline, visual characteristics of the subdermal and intradermal tissues in the body area of interest.

In accordance with a principal feature of the invention, a baseline image is first established prior to the initiation of the procedure to be monitored. In a non-limiting example, the administration of a radiographic contrast agent may be monitored. After the baseline image is obtained, detector 13 continues to collect NIR energy from the body area of interest and generates additional images of the area of interest during the procedure. Continued monitoring of the NIR images of the subdermal and intradermal tissues permits the determination of the state of the infusion process. These images are compared to the baseline image to determine changes near the vasculature in the body area of interest that evidence extravasation or infiltration.

An extravasated or infiltrated fluid or substance near a vein or artery is detected by observing the differences in the apparent image densities of the baseline vascular structure and infused substances in the area of interest. The image of the extravasated or infused fluid or substance may be darker or lighter depending on the relative absorbance of the NIR light as compared to the surrounding tissue and vasculature. Darker images are formed where the infusion fluid or substance absorbs or attenuates the NIR light more than the normal tissues as established in the baseline image.

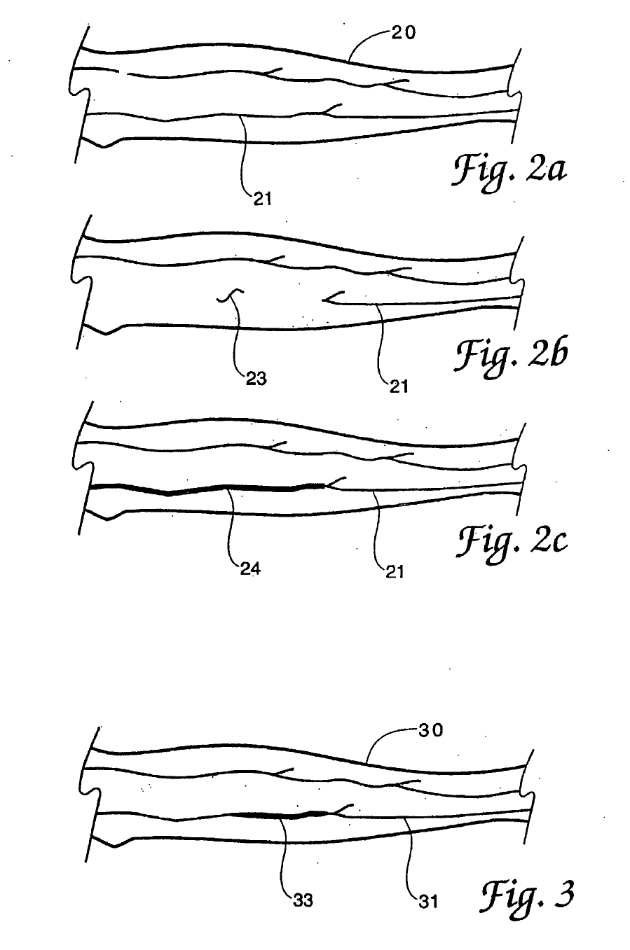

To illustrate the foregoing, reference is now made to FIGS. 2a-c. FIG. 2a shows a drawing of a baseline image of a forearm 20 of a patient as in FIG. 1. FIG. 2b shows the baseline image after a substance transparent to NIR is inserted into vein 21 of the patient. FIG. 2c shows the baseline image of FIG. 2a after a substance absorbent of the NIR is inserted into vein 21. The extravasation or infiltration of a contrast agent is determined either by the appearance of a lighter region 23 (FIG. 2b) or darker region 24 (FIG. 2c), depending on the transmissivity or absorbance of the infused substance, as compared to the baseline images created for the surrounding subdermal tissues prior to infusion. The representations of the extravasated or infiltrated substances can be any such size and shape depending on a number of factors including the volume and rate of substance penetration, the anatomy of the patient, the health of the patient, the size and location of the cannulation tool, among others. The illustrations in FIGS. 2a-b, therefore, do not limit the invention in terms of specifying the various sizes and shapes of detected substances.

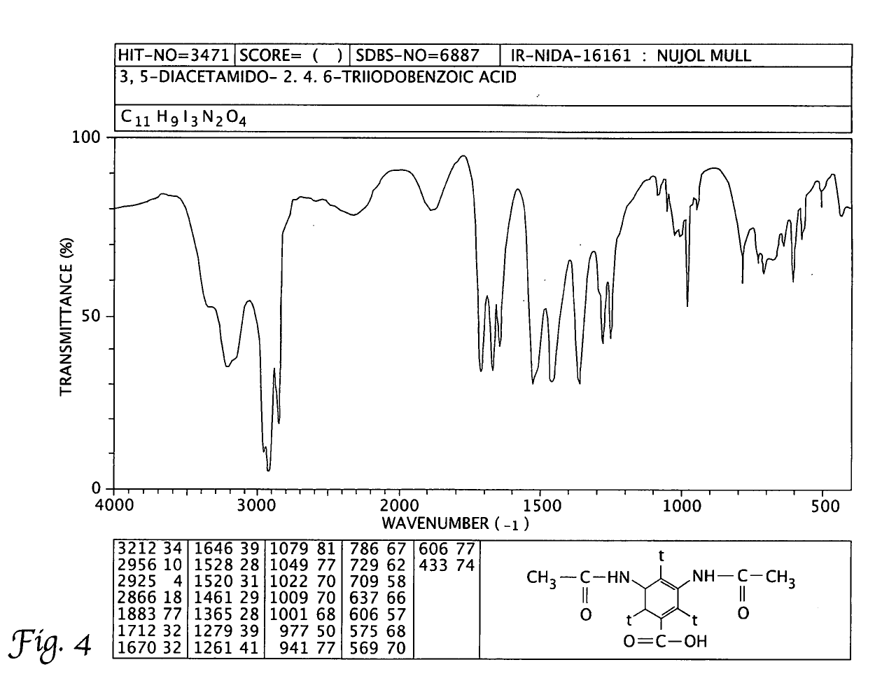

FIG. 3 shows schematically the forearm 30 of a patient after the IV instrument (15 of FIG. 1) is removed from vein 31, illustrating the presence of infiltrated blood 33 around vein 31. Infiltration of blood components is determined by a darkened region image because blood attenuates the NIR light more than the surrounding tissue. Again, the illustration of FIG. 3 does not limit the invention in terms of specifying the various sizes and shapes of detected blood.

The method taught by the invention can therefore be easily used to detect the occurrence of an infiltration and/or extravasation in real-time in a particular body area of interest. According to the invention, the generated images of the subdermal or intradermal tissues may be displayed to the practitioner on a video monitor, heads-up display or other presentation modality. Extravasation and/or infiltration in the body area of interest may be detected, optically imaged and distinguished in real-time during a procedure without obstruction or interruption of the procedure, and during minor movement of a patient without requiring re-imaging.

In further embodiments of the invention, near-infrared radiation (e.g., in the range of 0.7 μm-3.0 μm) and mid-infrared radiation (e.g., in the range of 3.0 μm-6.0 μm, and up to 20 μm) may be used to visualize the presence of extravasation of radiographic or other contrast media (as contrast media absorbs photonic energy in that band), blood, medications, and other fluids in subdermal tissues of human beings and other mammals. In addition, infrared radiation may be used to visualize the placement of vein access devices so that the risk of extravasation is minimized. It may also be used to visualize the extent and location of an extravasation or infiltration of fluids into tissue.

The invention may be used with an automated extravasation detector, such as that described in U.S. Patent Application Publication 2003/0036713A1 to Bouton et al. After an alarm or other means indicates that an injection has been terminated or is paused, the invention would be used to inspect the injection site to confirm or monitor the presence and extent of fluid extravasation. Alternatively, the invention may be used to monitor the injection in real-time, and provide a visible display to an operator to monitor the status of the injection as it occurs. Currently, clinicians palpate the injection site, in conjunction with visual inspection, to test for the presence of extravasate fluid. If the amount of fluid extravasate is less than the amount differentiable via palpation, the clinician has no good method, other than additional x-ray imaging or surgical intervention, to determine if the alarm was caused by a real extravasation or if the detector produced a false alarm (false positive detection.) The device could be used to measure and judge the amount of extravasation, the extent, the location, and the extent of likely tissue damage or adverse effects.

Additionally, the device may be used by clinicians while inserting needles or catheters for IV access procedures. If the patient's vein is difficult to find, or if there is compromised access, the clinician may use infrared imaging to readjust the needle or catheter, move the IV access location to a different location or vein, adjust fluid delivery parameters to minimize the risk of extravasation, increase the detection sensitivity of an extravasation detection device, more closely monitor the patient during the injection, and/or inspect the injection site visually, by touch, and with infrared imaging after the injection to check for extravasation.

Imaging contrast media has near-infrared absorption characteristics that are favorable for detecting the presence of that media in tissue using near-infrared radiation. It is known that ionic and non-ionic contrast media have a spectral peak or absorption around 1660 cm⁻¹ (approximately 6 μm), as well as other peaks in the region of 2.5 to 20 μm. The mid-infrared region provides for “finger print” fundamental band absorption of basic molecular bonds, including water, lipids, proteins, nucleic acids, and carbohydrates in tissues, as well as absorption of molecular bonds for administered fluids and medications. (See Guo et al., “Laser-Based Mid-Infrared Reflectance Imaging Of Biologic Tissues”, Opt. Express 12, 208-219 (2004) incorporated herein by reference) Imaging in this region may be used to distinguish the presence of extravasate contrast media in tissue, including extravasate near an injection site and below or within the skin, fat layer, or within the outer muscle strata of tissue. Using narrow band imaging and/or narrow bin, high frequency resolution, Fourier Transform Infrared (FTIR) measurements, it may be possible to detect the subdermal presence of specific fluids or medications if their infrared spectra is known by tuning the infrared measurements to the specific bands of interest. FIG. 4 shows the mid-infrared spectrum of diatrizoic acid, an ingredient in some contrast agents.

Some previous work in the literature has focused on the use of infrared spectroscopy for detecting surface and depth profiling of topical chemicals applied to the skin. (See Notingher, I, “Mid-Infrared In Vivo Depth Profiling Of Topical Chemicals On Skin,” Skin Research and Technology, 10: 113-121 (2004), incorporated herein by reference) There is, however, no known prior work on detecting the presence of subdermal material. In the mid-infrared region, tissue is generally more absorptive, however, with less scattering due to the longer wavelengths, as compared to near infrared imaging (NIR).

The IR spectrum for benzene, C₆H₆, a common chemical structure within compounds in imaging contrast agents, has only four prominent bands because it is a very symmetric molecule. Every carbon has a single bond to a hydrogen. Each carbon is bonded to two other carbons and the carbon-carbon bonds are alike for all six carbons. The molecule is planar. The aromatic CH stretch appears at 3100-3000 cm-1. There are aromatic CC stretch bands (for the carbon-carbon bonds in the aromatic ring) at about 1500 cm-1. Two bands are caused by bending motions involving carbon-hydrogen bonds. The bands for CH bends appear at approximately 1000 cm-1 for the in-plane bends and at about 675 cm-1 for the out-of-plane bend.

Extravasate contrast media may produce additional infrared absorption in these bands, including the near-infrared region at approximately 6000 cm⁻¹ (1660 nm), and thus may be detected using spectral infrared imaging techniques. The thought here is that, because blood is mixed with the extravasate, when a leak occurs a large percentage of the extravasate will absorb NIR because of the presence of the blood.

As a particular example, some Ionic CT contrast media contains diatrizoic acid compounds (3,5-diacetamido-2,4,6-triiodobenzoic acid) or amidotrizoic acid compounds which have a unique infrared spectra. For diatrizoic acid there are infrared absorption peaks near 1500 cm⁻¹ due to the carbon-carbon bonds in the central benzene ring, as well as other absorption peaks near the 1660, 1370, and 3200 cm⁻¹ bands. Infrared spectroscopy may be used to view the region around the injection site of the contrast media and to determine changes in absorption in these bands, as a way to detect an increase in the local tissue concentration of the contrast agent which may be an indication of a fluid extravasation.

Blood hemoglobin and other blood components also have characteristic infrared absorption spectra. The absorbance spectra for hemoglobin species, including oxy-, deoxy-, and carboxy- in the mid-infrared region have absorption bands centered at approximately 3280, 3080, 2964, 1653, 1541, 1456, 1396, 1302, 1248, and 1105 cm⁻¹. (See Kuenstner J T, et al., “Spectrophotometry of Human Hemoglobin in the Midinfrared Region,” Biospectroscopy, Vol. 3, Issue 3, 225-232 (1997), incorporated herein by reference) Decreases in absorption in these bands may indicated displacement or dilution of blood components by extravasate media near the injection area.

Also, it is possible to use similar extravasation detection and monitoring techniques based on near infrared imaging, based on the specific absorption characteristics of tissue and extravasate.

Mid-infrared imaging in the range of 3.0 to 6.0 μm or greater may also be used to image subsurface and surface temperature or emission differences of tissue and injected fluids as well as absorption effects due to injected contrast agent or medication and displaced blood. When imaging contrast media is administered to a patient, there may be significant temperature differences between the media and the surrounding tissue near the venous access region. (See U.S. Pat. No. 6,375,624 to Uber et al., “Extravasation Detector Using Microwave Radiometry,” incorporated herein by reference). Contrast media is typically heated. The initial injection volume of media, however, is typically cooler than body temperature, due to dwell time before delivery into the patient. If an extravasation is due to initial puncture of the injected vessel, or if the delivery needle or catheter has been inserted incorrectly so that it delivers fluid outside of the vessel and into the surrounding tissue, mid-infrared imaging may be used to detect the surface and subdermal temperature changes induced by the injected fluid. For non-heated fluids, such as with a typical IV or drug infusion, the ability to monitor for extravasation via mid-infrared visualization will be more pronounced, as the temperature difference will be greater than if the fluid was heated.

The method described above may also employ the use of infrared detector goggles, an infrared source, and a screen that presents the infrared energies that are absorbed by blood and contrast agent. Additional embodiments are possible through various combinations of optical source, source coupling or projection means, imaging device, and display device. Helpful in this regard is the disclosure in U.S. Patent Application Publication 2004/0171923A1 to Kalafut et al., “Devices, Systems And Methods For Improving Vessel Access,” incorporated herein by reference.

Also note that it may be possible to use other imaging means for infiltration detection in addition to continuous spectra infrared spectroscopy, including discrete spectra measurements, fluorescence spectroscopy, infrared Raman spectroscopy, multi-wavelength combined measurements, and combinations thereof. In addition, note that a variety of illumination modes may be used, such as reflection or transillumination, and that a variety of optical sources and imagers may be used, provided that they operate over the wavelengths of interest.

The invention therefore provides a system and a method for real-time visualization and detection of extravasated and/or infiltrated fluids and substances, including blood, that occur near the cannulation site of an injection. It is understood that modifications to the invention may be made as might occur to one with skill in the field of the invention within the scope of the appended claims. Consequently, all embodiments contemplated hereunder that achieve the objectives of the invention have not been shown in complete detail.

Several embodiments and related aspects for carrying out the invention have been set forth in detail according to the Patent Act. Persons of ordinary skill in the art to which this invention pertains may nevertheless recognize alternative ways of practicing the invention without departing from the spirit of the following claims. Consequently, all changes and variations that fall within the literal meaning, and range of equivalency, of the claims are to be embraced within their scope. Persons of such skill will also recognize that the scope of the invention is indicated by the claims rather than by any particular example or embodiment discussed in the foregoing description.

What is claimed is:

1. A method for real-time visualization and detection of extravasted and/or infiltrated fluids and substances, including blood, that occur near the cannulation site of an injection, the method comprising the steps of:
(a) providing a light source in the wavelength range of about 0.3 to about 1.0 microns;
(b) illuminating a portion of the body near the intended site of an injection in at least one of a reflection mode and a transillumination mode;
(c) providing a detector sensitive to light in said wavelength range of said light source for receiving said light transmitted from said portion of the body;
(d) selectively filtering the light received by said detector using a filter having a narrow passband centered substantially on at least one wavelength in said wavelength range of said light source;
(e) generating a first image from the filtered light to provide a baseline image of the subdermal and intradermal tissues in said portion of the body;
(f) generating at least one additional image of said portion of the body during a procedure that includes an injection at said body portion; and
(g) comparing said at least one additional image with said baseline image to detect changes near the vasculature in said portion of the body to detect extravasation or infiltration in said portion of the body.

2. The method of claim 1 wherein said detector includes an image intensifier tube, photomultiplier tube, a photo diode, a charge coupled device, or a CMOS.

3. The method of claim 1 wherein said step of filtering said detected energy is performed using a filter having a narrow passband centered substantially on at least one wavelength selected from the group consisting of 0.32, 0.345, 0.41, 0.43, 0.455, 0.54, 0.56, 0.58, 0.7, 0.76 microns.

4. A method for monitoring a medical procedure that includes an injection procedure at a portion of the body, the method comprising the steps of:
(a) providing a light source in the wavelength range of about 0.3 to about 1.0 microns;
(b) illuminating a portion of the body near the intended site of an injection in at least one of a reflection mode and a transillumination mode;
(c) providing a detector sensitive to light in said wavelength range of said light source for receiving said light transmitted from said portion of the body;
(d) selectively filtering the light received by said detector using a filter having a narrow passband centered substantially on at least one wavelength in said wavelength range of said light source;
(e) generating a series of images from the filtered light of the subdermal and intradermal tissues in said portion of the body during the medical procedure; and
(f) comparing said images to observe changes near the vasculature in said portion of the body and to detect extravasation and/or infiltration in said portion of the body.

5. The method of claim 4 wherein said detector includes an image intensifier tube, photomultiplier tube, a photo diode, a charge coupled device, or a CMOS.

6. The method of claim 4 wherein said step of filtering said detected energy is performed using a filter having a narrow passband centered substantially on at least one wavelength selected from the group consisting of 0.32, 0.345, 0.41, 0.43, 0.455, 0.54, 0.56, 0.58, 0.7, 0.76 microns.
